(19) United States
(12) Patent Application Publication
(10) Pub. No.: US 2025/0268554 A1
(43) Pub. Date: Aug. 28, 2025

(54) INFORMATION PROCESSING APPARATUS, RADIOGRAPHY SYSTEM, INFORMATION PROCESSING METHOD, AND PROGRAM

(71) Applicant: FUJIFILM CORPORATION, Tokyo (JP)

(72) Inventors: Hisatsugu HORIUCHI, Tokyo (JP); Yoshinori ANDO, Tokyo (JP); Tatsunari HOKIGUCHI, Tokyo (JP)

(21) Appl. No.: 19/048,967

(22) Filed: Feb. 10, 2025

(30) Foreign Application Priority Data
Feb. 22, 2024 (JP) 2024-025788

(51) Int. Cl. A61B 6/00; A61B 3/10; A61B 6/46; G01S 17/894

(52) U.S. Cl. CPC A61B 6/547; A61B 3/10; A61B 6/463; G01S 17/894

(57) ABSTRACT

Provided are an information processing apparatus, a radiography system, an information processing method, and a program capable of supporting determination of whether or not a specific part of a subject is included in an irradiation range of radiation. The information processing apparatus includes at least one processor. The processor is configured to: recognize a specific part of a subject from an optical image obtained by capturing the subject; derive a distance to the recognized specific part using a distance image obtained by capturing the subject; and derive an irradiation range of radiation at a position corresponding to the derived distance in an irradiation axis direction.

### CROSS-REFERENCE TO RELATED APPLICATIONS

The present application claims priority under 35 U.S.C. § 119 to Japanese Patent Application No. 2024-025788, filed on Feb. 22, 2024. The above application is hereby expressly incorporated by reference, in its entirety, into the present application.

### BACKGROUND

### 1. Technical Field

The disclosed technology relates to an information processing apparatus, a radiography system, an information processing method, and a program.

### 2. Description of the Related Art

Regarding a technology of controlling an irradiation range of radiation in a radiography apparatus, the following technology is known. For example, in description of JP2021-191403A, whether or not a structure of a specific shape having a transmittance of radiation lower than that of a subject is present in an imaging region of a radiography apparatus is specified based on the specific shape, and in a case where the structure is present, control is performed such that a region excluding the structure is the imaging region.

JP2017-534401A describes a pre-X-ray exposure control device including a subject detection unit, a subject model unit, an interface unit, a processing unit, and a display unit. The subject detection unit detects subject data of a subject to be exposed. The subject model unit provides a subject model and refines the subject model based on the subject data to obtain a refined subject model. The interface unit provides setting data of an X-ray unit to be used for exposing the subject. The processing unit calculates a virtual X-ray projection image based on the refined subject model and the provided setting data. The display unit displays the virtual X-ray projection image. The setting data is a collimation parameter of an X-ray unit to be used for exposing a partial region of the subject. The subject detection unit detects a position of an anatomical landmark of the subject and detects an orientation of the subject based on the position of the anatomical landmark.

### SUMMARY

In a case where a radiation image is captured for a subject, an irradiation range of radiation may be adjusted such that a specific part of the subject is not irradiated with the radiation. For example, in a videofluoroscopic examination of swallowing using a radiography apparatus, an irradiation range of radiation is adjusted such that an eyeball of the subject is not irradiated with the radiation.

In the adjustment of the irradiation range of the radiation, for example, the irradiation range of the radiation is confirmed by irradiating the subject with collimated light for displaying the irradiation range of the radiation. However, in a case where an uneven part (for example, nose and mouth) of the subject is included in the irradiation range of the collimated light, an edge position of the collimated light is unclear. As a result, it is difficult to determine whether or not a specific part of the subject, which is to be avoided from the irradiation with the radiation, is included in the irradiation range of the radiation.

The disclosed technology has been made in view of the above-described points, and an object of the disclosed technology is to support a determination of whether or not the specific part of the subject is included in the irradiation range of the radiation.

According to the disclosed technology, there is provided an information processing apparatus comprising at least one processor. The processor is configured to: recognize a specific part of a subject from an optical image obtained by capturing the subject; derive a distance to the recognized specific part using a distance image obtained by capturing the subject; and derive an irradiation range of radiation at a position corresponding to the derived distance in an irradiation axis direction.

The processor may be configured to display information indicating the derived irradiation range on a display screen, by superimposing the information on the optical image. The processor may be configured to display the optical image and a radiation image side by side. The processor may be configured to issue an alert in a case where the recognized specific part is included in the derived irradiation range.

The processor may be configured to control the irradiation range of the radiation such that the recognized specific part is not included in the irradiation range of the radiation in accordance with a command, in a case where the recognized specific part is included in the derived irradiation range. The processor may be configured to control the irradiation range of the radiation by controlling an opening amount of an opening portion, through which the radiation passes, of a collimator that forms the opening portion. The specific part may be an eyeball of the subject.

A radiography system according to the disclosed technology includes the above-described information processing apparatus, an optical camera that generates an optical image, a distance sensor that generates a distance image, and a radiation source unit that emits radiation.

The optical camera may be disposed outside an irradiation range of the radiation emitted from the radiation source unit. The optical camera may be disposed at a position closer to an irradiation axis of the radiation emitted from the radiation source unit with respect to the distance sensor. The distance sensor may be a ToF camera or a stereo camera.

An information processing method according to the disclosed technology is a method executed by at least one processor included in an information processing apparatus, the method including a process comprising: recognizing a specific part of a subject from an optical image obtained by capturing the subject; deriving a distance to the recognized specific part using a distance image obtained by capturing the subject; and deriving an irradiation range of radiation at a position corresponding to the derived distance in an irradiation axis direction.

A program according to the disclosed technology is a program that causes at least one processor included in an information processing apparatus, to execute a process comprising: recognizing a specific part of a subject from an optical image obtained by capturing the subject; deriving a distance to the recognized specific part using a distance image obtained by capturing the subject; and deriving an irradiation range of radiation at a position corresponding to the derived distance in an irradiation axis direction.

According to the disclosed technology, it is possible to support the determination of whether or not the specific part of the subject is included in the irradiation range of the radiation.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a radiography system according to an embodiment of disclosed technology;

FIG. 2 is a view showing an example of a configuration of a radiography apparatus according to the embodiment of the disclosed technology;

FIG. 3 is a diagram showing an example of a relationship between an irradiation range of radiation and an imaging range of an optical camera according to the embodiment of the disclosed technology;

FIG. 4A is a diagram showing a positional relationship between the optical camera and a distance sensor according to the embodiment of the disclosed technology;

FIG. 4B is a diagram showing the positional relationship between the optical camera and the distance sensor according to the embodiment of the disclosed technology;

FIG. 5 is a diagram showing an example of a hardware configuration of an information processing apparatus according to the embodiment of the disclosed technology;

FIG. 6 is a functional block diagram showing an example of a functional configuration of the information processing apparatus according to the embodiment of the disclosed technology;

FIG. 7 is a view for describing a method of deriving an irradiation range of radiation according to the embodiment of the disclosed technology;

FIG. 8 is a diagram showing an example of a display form of information indicating the irradiation range of the radiation according to the embodiment of the disclosed technology;

FIG. 9 is a diagram showing an example of a relationship between an irradiation range of radiation and an imaging range of an optical camera;

FIG. 10 is a flowchart showing an example of a flow of processing performed by executing a processing program by a CPU of the information processing apparatus according to the embodiment of the disclosed technology;

FIG. 11 is a functional block diagram showing an example of a functional configuration of an information processing apparatus according to another embodiment of the disclosed technology;

FIG. 12 is a flowchart showing an example of a flow of processing performed by executing a processing program by a CPU of the information processing apparatus according to another embodiment of the disclosed technology;

FIG. 13 is a functional block diagram showing an example of the functional configuration of the information processing apparatus according to another embodiment of the disclosed technology;

FIG. 14 is a perspective view showing an example of a configuration of a collimator according to the embodiment of the disclosed technology; and

FIG. 15 is a flowchart showing an example of a flow of processing performed by executing a processing program according to another embodiment of the disclosed technology.

### DETAILED DESCRIPTION

An example of embodiments of the disclosed technology will be described below with reference to the drawings. It is noted that the same or equivalent components and portions in the drawings are assigned by the same reference numerals, and the overlapping description will be omitted.

### First Embodiment

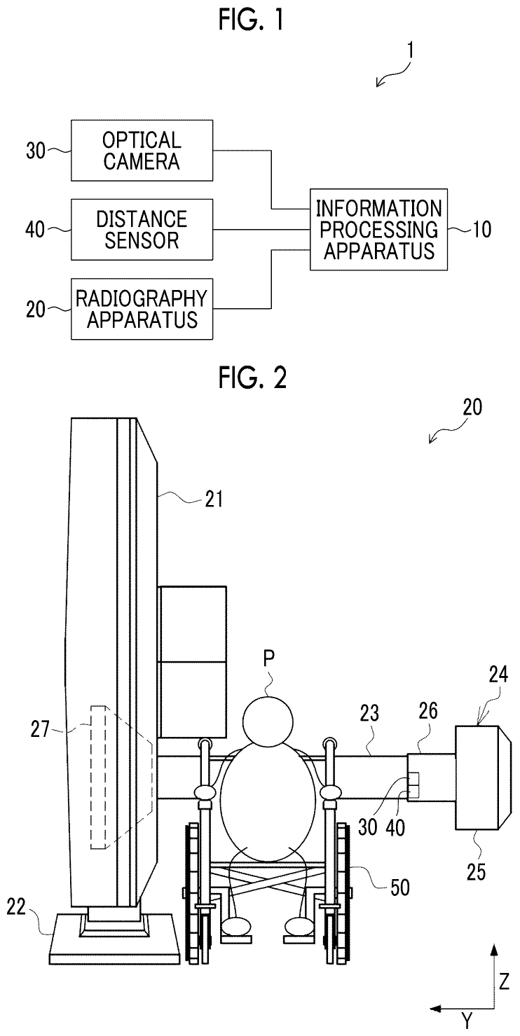

FIG. 1 is a diagram showing an example of a configuration of a radiography system 1 according to an embodiment of the disclosed technology. The radiography system 1 includes an information processing apparatus 10, a radiography apparatus 20, an optical camera 30, and a distance sensor 40.

FIG. 2 is a diagram showing an example of the configuration of the radiography apparatus 20. The radiography apparatus 20 is capable of imaging a radiation image using radiation such as X-rays. The radiography apparatus 20 has an imaging table 21. The imaging table 21 is supported on the floor surface by a stand 22. A radiation source unit 24 is attached to the imaging table 21 via a support column 23. The radiation source unit 24 is configured to include a radiation source 25 and a collimator 26. The imaging table 21 has a built-in radiation detector 27.

The radiation source 25 has a radiation tube (not shown) that emits radiation. The collimator 26 limits an irradiation range of the radiation emitted from the radiation tube. The radiation detector 27 has a plurality of pixels that generate signal charges in accordance with the radiation that has been transmitted through a subject P. The radiation detector 27 is called a flat panel detector (FPD).

The radiation source unit 24 is capable of reciprocating together with the support column 23 along a long side direction of the imaging table 21 by a moving mechanism (not shown) such as a motor. The radiation detector 27 is capable of reciprocating along the long side direction of the imaging table 21 in conjunction with the movement of the radiation source unit 24. The imaging table 21 and the support column 23 can be rotated between a standing state shown in FIG. 2 and a supine state (not shown) by a rotation mechanism (not shown) such as a motor. In the supine state, the surface of the imaging table 21 is parallel to the floor surface, and the direction in which the support column 23 extends is perpendicular to the floor surface. On the other hand, in the standing state, the surface of the imaging table 21 is perpendicular to the floor surface, and the direction in which the support column 23 extends is parallel to the floor surface. In the standing state, it is possible to perform fluoroscopy on the subject P in a wheelchair 50 as shown in FIG. 2. In the following description, as shown in FIG. 2, a case (LR imaging) where the radiation is emitted from a side of the subject P to capture the radiation image will be described as an example. In addition, an irradiation axis direction of the radiation is referred to as a Y direction, a vertical direction is referred to as a Z direction, and a front-back direction of the subject P is referred to as an X direction.

The optical camera 30 is an imaging apparatus that can generate an optical image (RGB image). An optical image of the subject P is captured by the optical camera 30. The optical camera 30 is attached to the radiation source unit 24. In FIG. 2, a case where the optical camera 30 is attached to a housing of the collimator 26 is shown, but the optical camera 30 may be attached to a housing of the radiation source 25.

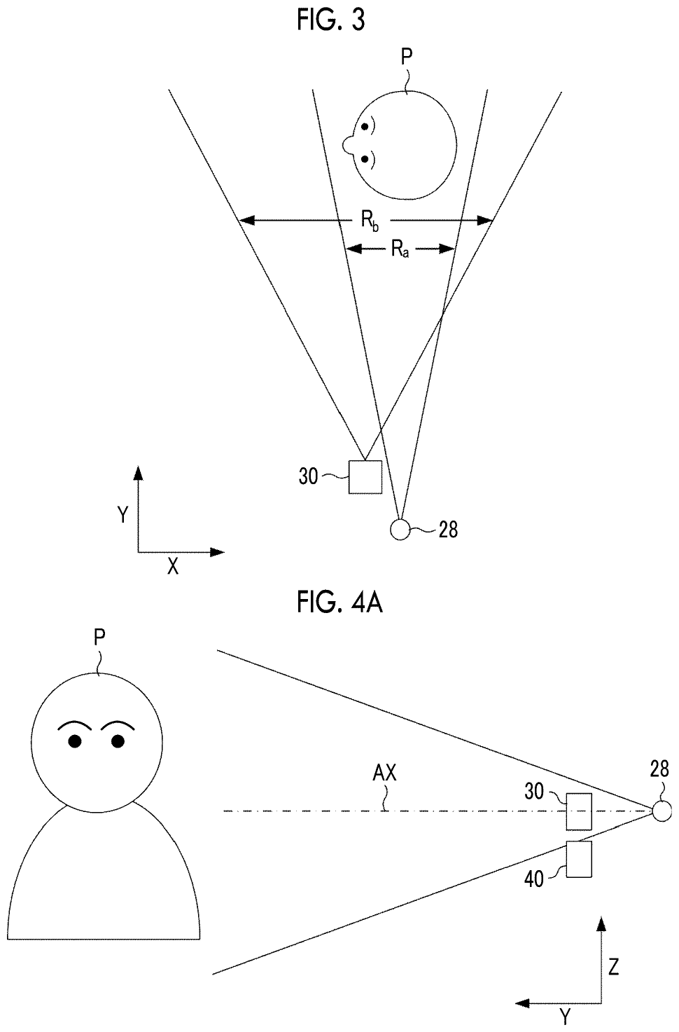

FIG. 3 is a diagram showing an example of a relationship between an irradiation range Ra of the radiation emitted from a radiation tube 28 and an imaging range Rb of the optical camera 30. In order to avoid the optical camera 30 from being captured on the radiation image, the optical camera 30 is disposed outside the irradiation range Ra of the radiation. Therefore, an optical axis of the optical camera 30 and an irradiation axis of the radiation are parallel to each other but are not matched. The entire irradiation range Ra of the radiation at a position of the subject P is included in the imaging range Rb of the optical camera 30.

The distance sensor 40 is a device that can generate a distance image showing a distance to a surface of an object. A distance image of the subject P is captured by the distance sensor 40. The distance sensor 40 may be, for example, a time-of-flight (ToF) camera that generates a distance image with a ToF method using infrared light. In addition, the distance sensor 40 may be a stereo camera that generates a distance image based on a principle of triangulation using two cameras. The distance sensor 40 is attached to the radiation source unit 24, similarly to the optical camera 30. In FIG. 2, a case where the distance sensor 40 is attached to the housing of the collimator 26 is shown, but the distance sensor 40 may be attached to the housing of the radiation source 25.

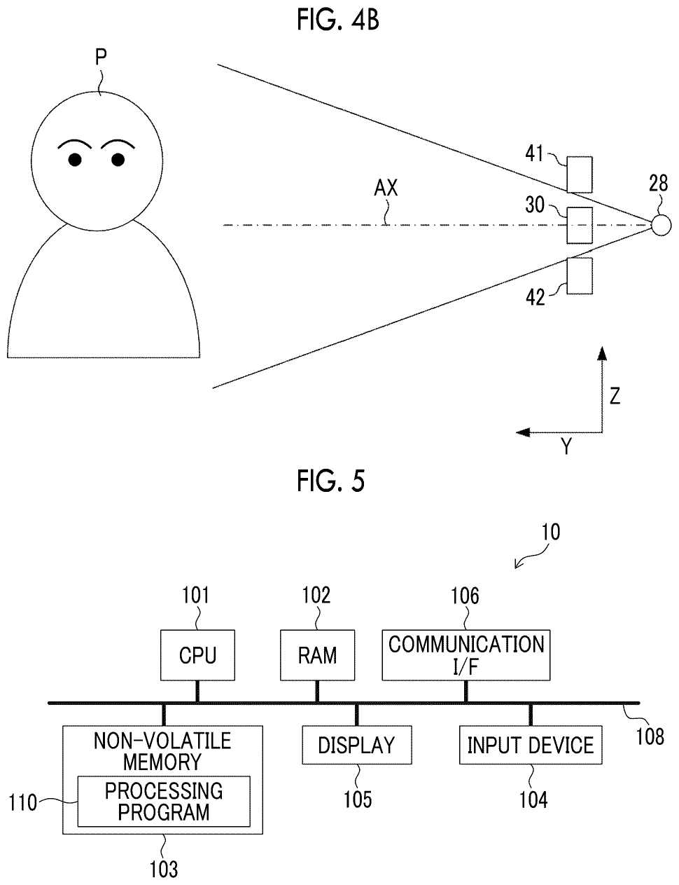

FIGS. 4A and 4B are diagrams showing a positional relationship between the optical camera 30 and the distance sensor 40, respectively. FIG. 4A shows a case where the distance sensor 40 is a ToF camera, and FIG. 4B shows a case where the distance sensor 40 is a stereo camera. The optical camera 30 is disposed at a position closer to an irradiation axis AX of the radiation emitted from the radiation tube 28 with respect to the distance sensor. In a case where the distance sensor 40 is a stereo camera having two cameras 41 and 42, as shown in FIG. 4B, the optical camera 30 may be disposed between the two cameras 41 and 42 constituting the distance sensor 40. It is preferable that a position of the optical axis of the optical camera 30 and a position of the irradiation axis AX of the radiation match with each other in the Z direction (vertical direction).

The information processing apparatus 10 has a function of deriving an irradiation range of radiation at a position on an irradiation axis where a specific part of the subject P is present, based on the optical image of the subject P output from the optical camera 30 and the distance image of the subject P output from the distance sensor 40. The “specific part” is a predetermined part as a part to be avoided from irradiation with radiation among parts of body of the subject P. In the following, a case where the specific part to be avoided from the irradiation with the radiation is an eyeball will be described as an example.

FIG. 5 is a diagram showing an example of a hardware configuration of the information processing apparatus 10. The information processing apparatus 10 includes a central processing unit (CPU) 101, a random-access memory (RAM) 102, a non-volatile memory 103, an input device 104 including a keyboard and a mouse, a display 105, and a communication interface 106. These pieces of hardware are connected to a bus 108.

The display 105 may be a touch panel display. The communication interface 106 is an interface for the information processing apparatus 10 to perform communication with the optical camera 30, the distance sensor 40, and the radiography apparatus 20. The communication method may be either wired or wireless. For the wireless communication, it is possible to apply a method conforming to existing wireless communication standards such as, for example, Wi-Fi (registered trademark) and Bluetooth (registered trademark).

The non-volatile memory 103 is a non-volatile storage medium such as a hard disk or a flash memory. A processing program 110 is stored in the non-volatile memory 103. The RAM 102 is a work memory for the CPU 101 to execute processing. The CPU 101 loads the processing program 110 stored in the non-volatile memory 103 into the RAM 102, and executes processing in accordance with the processing program 110. The CPU 101 is an example of a “processor” in the disclosed technology.

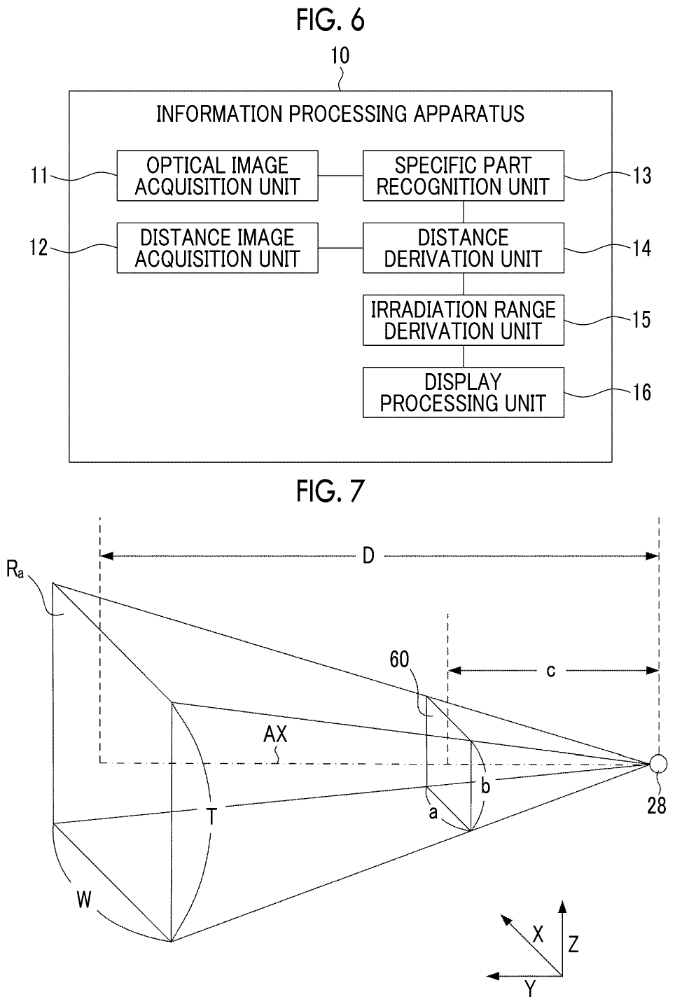

FIG. 6 is a functional block diagram showing an example of a functional configuration of the information processing apparatus 10. The information processing apparatus 10 includes an optical image acquisition unit 11, a distance image acquisition unit 12, a specific part recognition unit 13, a distance derivation unit 14, an irradiation range derivation unit 15, and a display processing unit 16. By executing the processing program 110 by the CPU 101, the information processing apparatus 10 functions as the optical image acquisition unit 11, the distance image acquisition unit 12, the specific part recognition unit 13, the distance derivation unit 14, the irradiation range derivation unit 15, and the display processing unit 16.

The optical image acquisition unit 11 acquires the optical image of the subject P output from the optical camera 30. The distance image acquisition unit 12 acquires the distance image of the subject P output from the distance sensor 40.

The specific part recognition unit 13 recognizes the eyeball, which is the specific part to be avoided from the irradiation with the radiation, among the parts of the body of the subject P from the optical image acquired by the optical image acquisition unit 11. The specific part recognition unit 13 recognizes the specific part (eyeball) from the optical image using a discriminator constructed by machine learning using the optical image, in which correct answer information is attached to the specific part, as training data.

The distance derivation unit 14 derives a distance to the specific part (eyeball) recognized by the specific part recognition unit 13 by using the distance image acquired by the distance image acquisition unit 12. More specifically, the distance derivation unit 14 transforms a coordinate position on the optical image where the specific part (eyeball) recognized by the specific part recognition unit 13 is present, into a coordinate position on the distance image. This coordinate transformation can be performed by geometric calculation based on installation positions of the optical camera 30 and the distance sensor 40. The distance derivation unit 14 derives the distance to the specific part (eyeball) from a pixel value at the coordinate position on the distance image in which the specific part (eyeball) is present.

The irradiation range derivation unit 15 derives an irradiation range of radiation at a position corresponding to the distance to the specific part (eyeball) derived by the distance derivation unit 14, in the irradiation axis direction (Y direction). FIG. 7 is a diagram for describing a method of deriving an irradiation range of radiation. The radiation emitted from the radiation tube 28 is limited to a range of an opening portion 60 of the collimator disposed in front of the radiation tube 28. Dimensions a and b of the opening portion 60 and a distance c from the radiation tube 28 to the opening portion 60 are known. A distance D from the radiation tube 28 to the specific part (eyeball) of the subject P can be acquired by performing correction in accordance with a positional relationship between the distance sensor 40 and the radiation tube 28, with respect to the distance to the eyeball derived by the distance derivation unit 14. The irradiation range derivation unit 15 derives the irradiation range of the radiation at the position corresponding to the distance to the eyeball, in the irradiation axis direction (Y direction), by calculating W represented by the following Expression (1) and T represented by the following Expression (2). That is, the irradiation range derivation unit 15 derives a rectangular region having a length of W in the X direction and a length of T in the Z direction with the irradiation axis AX of the radiation as a center, as the irradiation range of the radiation at the eyeball position of the subject P.

$$W = a \times D / c \qquad ( 1 )$$

$$T = b \times D / c \qquad ( 2 )$$

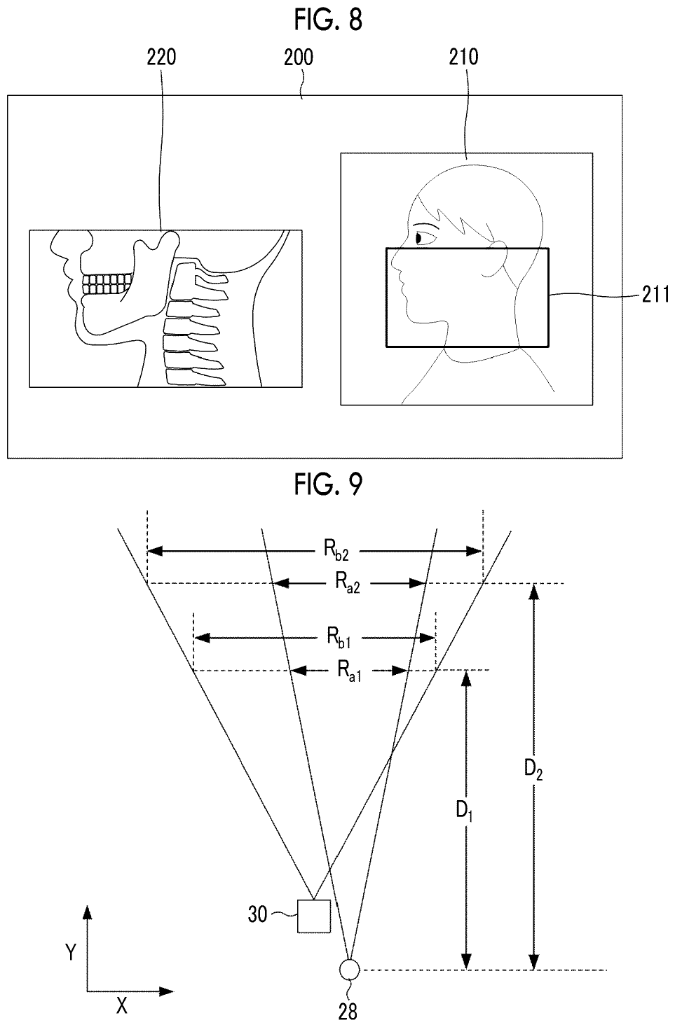

The display processing unit 16 displays information indicating the irradiation range of the radiation derived by the irradiation range derivation unit 15 on a display screen of the display 105, by superimposing the information on the optical image of the subject P acquired by the optical image acquisition unit 11. FIG. 8 is a diagram showing an example of a display form of information indicating an irradiation range of radiation displayed on a display screen 200. The display screen 200 may be a screen of a console installed outside an imaging room or may be a display screen of a monitor cart provided in the imaging room.

The display processing unit 16 displays a bounding box 211 as information indicating the irradiation range of the radiation (that is, the irradiation range of the radiation at the position corresponding to the distance to the eyeball) derived by the irradiation range derivation unit 15, in the irradiation axis direction (Y direction), on an optical image 210 of the subject P acquired by the optical image acquisition unit 11, by superimposing the information. As shown in FIG. 8, the display processing unit 16 may display a radiation image 220 and the optical image 210 of the subject P side by side. The radiation image 220 may be a fluoroscopic image (video) acquired by fluoroscopy using the radiography apparatus 20. The optical image 210 and the radiation image 220 are displayed on the display screen 200 in real time.

FIG. 9 is a diagram showing an example of a relationship between an irradiation range of the radiation emitted from the radiation tube 28 and an imaging range of the optical camera 30. As described above, since the optical axis of the optical camera 30 and the irradiation axis of the radiation are not matched, a position and a size of the irradiation range of the radiation within the imaging range of the optical camera 30 change in accordance with a distance from the radiation tube 28. That is, a position and a size of an irradiation range Rₐ₁ of radiation at a position at a distance D₁ from the radiation tube 28 within an imaging range R_{b1} of the optical camera 30 at the same position are different from a position and a size of an irradiation range Rₐ₂ of radiation at a position at a distance D₂ from the radiation tube 28 within an imaging range R_{b2} of the optical camera 30 at the same position.

In consideration of the above-described points, in a case where the bounding box 211 is displayed on the optical image 210 in a superimposed manner, the display processing unit 16 derives a position and a size of the bounding box 211 on the optical image 210, based on the distance (distance D from the radiation tube 28 to the eyeball of the subject P) derived by the distance derivation unit 14. The position and size of the bounding box 211 on the optical image 210 can be derived by performing geometric calculation in consideration of a positional relationship between the radiation tube 28 and the optical camera 30.

A capturer of the radiation image can ascertain whether or not the specific part (eyeball) of the subject P is included in the irradiation range of the radiation by confirming the display screen 200 shown in FIG. 8 as an example.

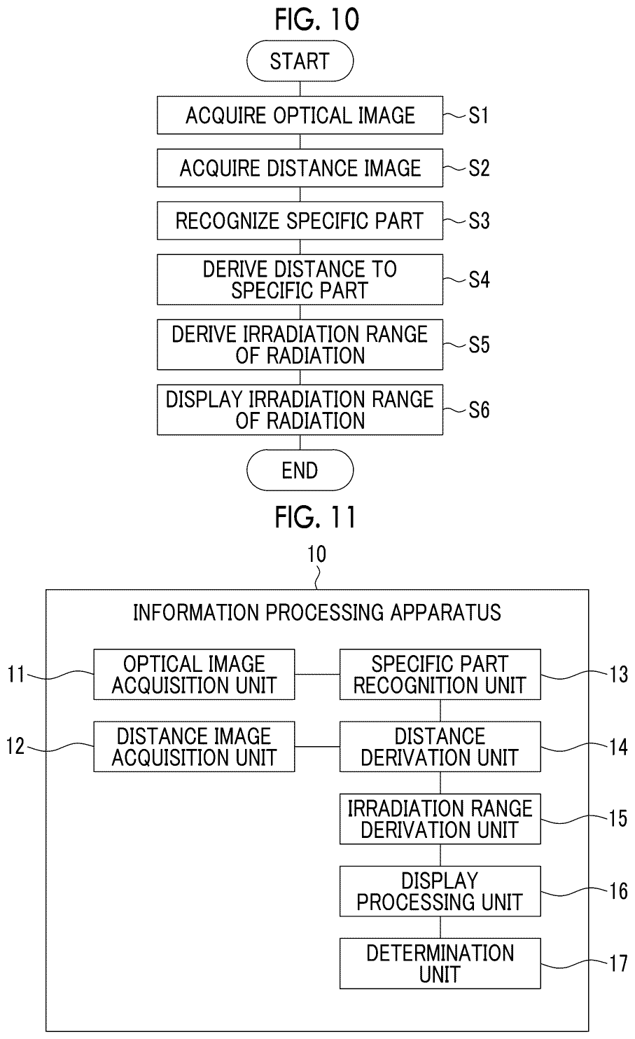

FIG. 10 is a flowchart showing an example of a flow of processing performed by the CPU 101 executing the processing program 110.

In step S1, the optical image acquisition unit 11 acquires the optical image of the subject P output from the optical camera 30.

In step S2, the distance image acquisition unit 12 acquires the distance image of the subject P output from the distance sensor 40.

In step S3, the specific part recognition unit 13 recognizes the eyeball, which is the specific part to be avoided from the irradiation with the radiation, among the parts of the body of the subject P from the optical image acquired in step S1.

In step S4, the distance derivation unit 14 derives the distance to the specific part (eyeball) recognized in step S3 by using the distance image acquired in step S2.

In step S5, the irradiation range derivation unit 15 derives an irradiation range of radiation at a position corresponding to the distance to the specific part (eyeball) derived in step S4, in the irradiation axis direction (Y direction).

In step S6, the display processing unit 16 displays the information indicating the irradiation range of the radiation derived in step S5, on the display screen of the display 105 by superimposing the information on the optical image of the subject P acquired in step S1.

As described above, the information processing apparatus 10 according to the embodiment of the disclosed technology recognizes a specific part of a subject from an optical image obtained by capturing the subject, derives a distance to the recognized specific part using a distance image obtained by capturing the subject, derives an irradiation range of radiation at a position corresponding to the derived distance in an irradiation axis direction, and displays information indicating the derived irradiation range on a display screen by superimposing the information manner on the optical image.

In a case where a radiation image is captured for a subject, an irradiation range of radiation may be adjusted such that a specific part of the subject is not irradiated with the radiation. For example, in a videofluoroscopic examination of swallowing using a radiography apparatus, an irradiation range of radiation is adjusted such that an eyeball of the subject is not irradiated with the radiation.

In the adjustment of the irradiation range of the radiation, for example, the irradiation range of the radiation is confirmed by irradiating the subject with collimated light for displaying the irradiation range of the radiation. However, in a case where an uneven part (for example, nose and mouth) of the subject is included in the irradiation range of the collimated light, an edge position of the collimated light is unclear. As a result, it is difficult to determine whether or not a specific part of the subject, which is to be avoided from the irradiation, is included in the irradiation range of the radiation.

With the information processing apparatus 10 according to the embodiment of the disclosed technology, the irradiation range of the radiation at the position corresponding to the distance to the specific part of the subject in the irradiation axis direction is derived regardless of presence or absence of unevenness. Therefore, it is easy to determine whether or not the specific part is included in the irradiation range of the radiation. That is, with the information processing apparatus 10 according to the embodiment of the disclosed technology, it is possible to support the determination of whether or not the specific part is included in the irradiation range of the radiation.

In addition, as shown in FIGS. 4A and 4B, since the optical camera 30 is disposed at the position closer to the irradiation axis AX of the radiation with respect to the distance sensor 40, a parallax between the optical image and the radiation image can be reduced. Therefore, it is easy to perform registration in a case where the information indicating the irradiation range of the radiation is displayed by superimposing the information on the optical image.

### Second Embodiment

FIG. 11 is a functional block diagram showing an example of a functional configuration of the information processing apparatus 10 according to a second embodiment of the disclosed technology. The information processing apparatus 10 according to the second embodiment is different from the information processing apparatus 10 according to the above-described first embodiment in that a determination unit 17 is further provided.

The determination unit 17 determines whether or not the eyeball of the subject P, which is the specific part recognized by the specific part recognition unit 13, is included in the irradiation range of the radiation derived by the irradiation range derivation unit 15. In a case where the determination unit 17 determines that the eyeball of the subject P is included in the irradiation range of the radiation, the determination unit 17 issues an alert. The alert may be issued by, for example, display on the display 105, output of a voice, light emission of a lamp, or the like.

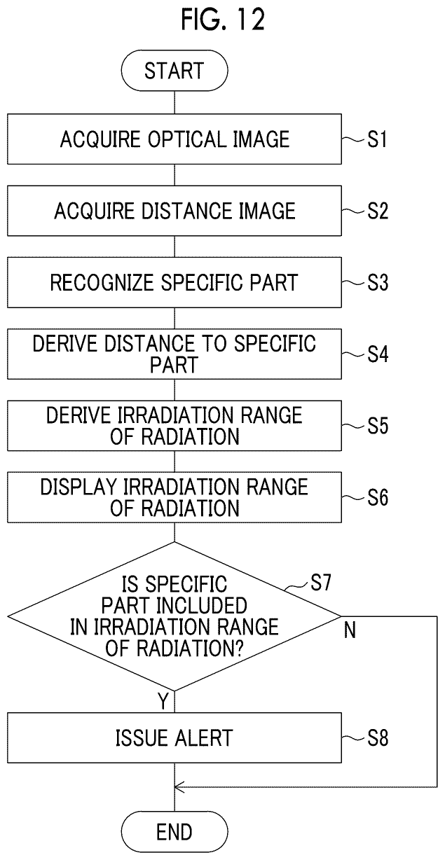

FIG. 12 is a flowchart showing an example of a flow of processing performed by executing the processing program 110 by the CPU 101 of the information processing apparatus 10 according to the second embodiment. Since the processing from step S1 to step S6 is the same as the processing according to the above-described first embodiment, the description thereof will not be shown.

In step S7, the determination unit 17 determines whether or not the specific part (eyeball) recognized in step S3 is included in the irradiation range of the radiation derived in step S5. In a case where it is determined that the specific part (eyeball) is included in the irradiation range of the radiation, the processing proceeds to step S8, and in a case where it is determined that the specific part (eyeball) is not included in the irradiation range of the radiation, the present routine ends.

In step S8, the determination unit 17 issues an alert to notify that the specific part (eyeball) is included in the irradiation range of the radiation.

With the information processing apparatus 10 according to the second embodiment, in a case where the specific part (eyeball) of the subject P is included in the irradiation range of the radiation, an alert is issued, so that it is possible to suppress a risk that the specific part (eyeball) is irradiated with the radiation.

### Third Embodiment

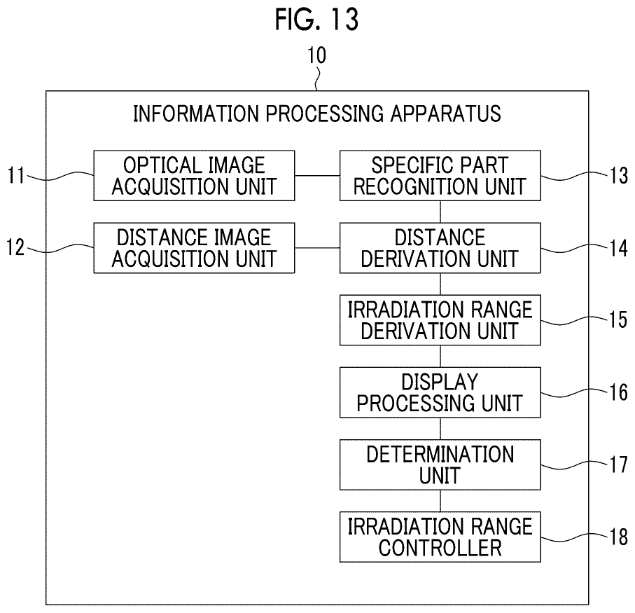

FIG. 13 is a functional block diagram showing an example of a functional configuration of the information processing apparatus 10 according to a third embodiment of the disclosed technology. The information processing apparatus 10 according to the third embodiment is different from the information processing apparatus 10 according to the above-described second embodiment in that an irradiation range controller 18 is further provided.

In a case where the eyeball of the subject P, which is the specific part recognized by the specific part recognition unit 13, is included in the irradiation range of the radiation derived by the irradiation range derivation unit 15, the irradiation range controller 18 controls the irradiation range of the radiation such that the specific part (eyeball) is not included in the irradiation range of the radiation in accordance with a command. The irradiation range controller 18 controls the irradiation range of the radiation by controlling an opening amount of an opening portion, through which the radiation passes, of the collimator 26 that forms the opening portion.

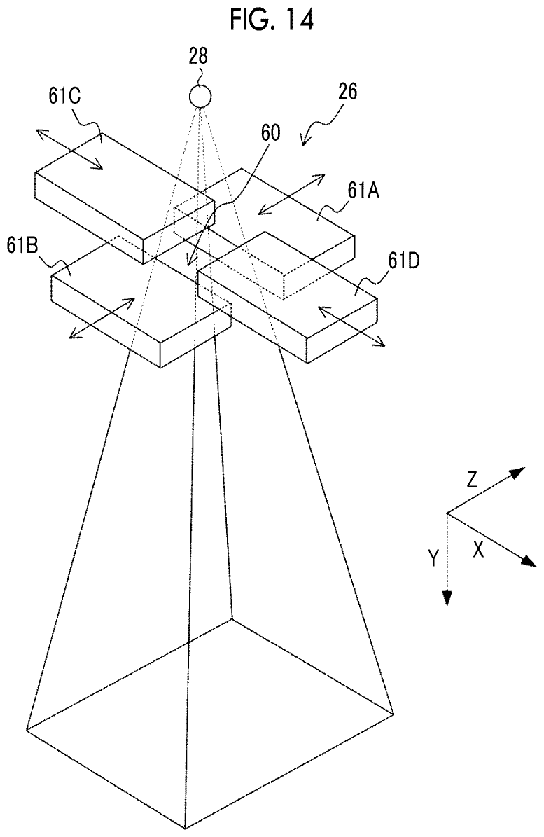

FIG. 14 is a perspective view showing an example of a configuration of the collimator 26. The collimator 26 includes four blades 61A, 61B, 61C, and 61D. The blades 61A to 61D are plate-like members made of a material that shields radiation, such as lead or tungsten. Each blade is disposed such that one side surface of the blade 61A and one side surface of the blade 61B face each other and one side surface of the blade 61C and one side surface of the blade 61D face each other. The opening portion 60 having a rectangular shape in plan view is formed in a region surrounded by the blades 61A to 61D. The radiation emitted from the radiation tube 28 passes through the opening portion 60 to be emitted to the subject P.

Each of the blades 61A to 61D can be moved by a linear motion mechanism (not shown) including a motor, a rack, and a pinion. In a case where the radiation source unit 24 is in a posture shown in FIG. 2, the blades 61A and 61B are movable in the Z direction, and the blades 61C and 61D are movable in the X direction. The irradiation range controller 18 controls an opening amount of the opening portion 60 by moving the blades 61A to 61D such that the specific part (eyeball) is not included in the irradiation range of the radiation. Movement amounts of the blades 61A to 61D for allowing the specific part (eyeball) to not be included in the irradiation range of the radiation can be derived from a positional relationship (distance) between the irradiation range of the radiation derived by the irradiation range derivation unit 15 and the specific part (eyeball) recognized by the specific part recognition unit 13.

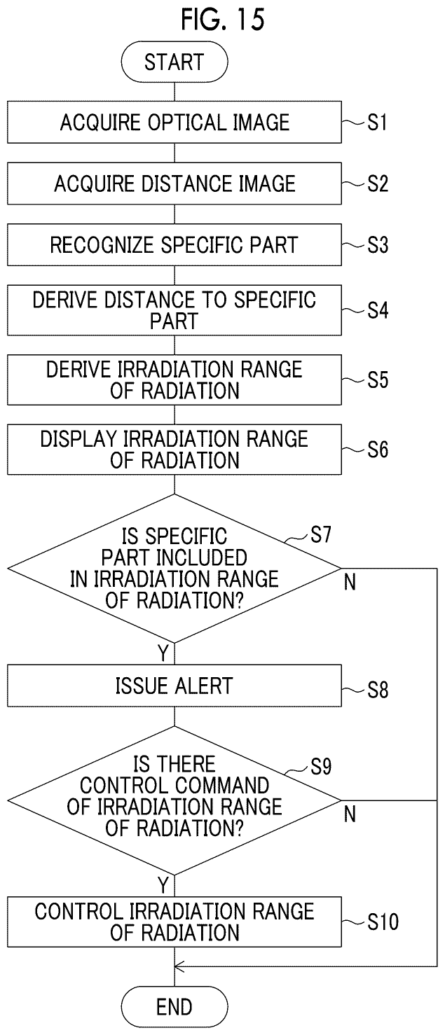

FIG. 15 is a flowchart showing an example of a flow of processing performed by executing the processing program 110 by the CPU 101 of the information processing apparatus 10 according to the third embodiment. Since the processing from step S1 to step S8 is the same as the processing according to the above-described second embodiment, the description thereof will not be shown.

In step S9, the irradiation range controller 18 determines presence or absence of a control command for controlling the irradiation range of the radiation. The control command of the irradiation range of the radiation is input to the information processing apparatus 10 by, for example, operating the input device 104 by a user. In a case where it is determined that there is the control command, the processing proceeds to step S10, and in a case where it is determined that there is no control command, the present routine ends.

In step S10, the irradiation range controller 18 controls the irradiation range of the radiation such that the specific part (eyeball) recognized in step S2 is not included in the irradiation range of the radiation. The irradiation range controller 18 controls the irradiation range of the radiation by controlling the opening amount of the opening portion 60 of the collimator 26.

With the information processing apparatus 10 according to the third embodiment, in a case where a specific part of the subject P is included in an irradiation range of radiation, the irradiation range of the radiation is controlled such that the part is not included in the irradiation range of the radiation. Therefore, it is possible to reduce work burden related to the adjustment of the irradiation range for avoiding the irradiation of the radiation to the specific part.

In the above description, a case where the “specific part” is the eyeball has been described as an example, but the disclosed technology is not limited to thereto. For example, a part to be avoided from the irradiation of the radiation, such as a reproductive gland of the subject and a hand of the capturer, can be referred to as a “specific part”. The disclosed technology can also be applied in a case where a part other than the eyeball is referred to as a “specific part”. In addition, the disclosed technology is not limited to the videofluoroscopic examination of swallowing, and can be applied to any examination using a radiography apparatus.

In addition, in the above description, a case where the specific part recognition unit 13 recognizes the specific part from the optical image by using the discriminator constructed by machine learning has been described as an example. However, the specific part may be directly recognized by using a result learned from the optical image, or may be secondarily recognized in consideration of an anatomical geometrical relationship from a part such as a joint that is easily extracted directly.

In each of the above-described embodiments, for example, as a hardware structure of processing units that execute various types of processing, such as the optical image acquisition unit 11, the distance image acquisition unit 12, the specific part recognition unit 13, the distance derivation unit 14, the irradiation range derivation unit 15, the display processing unit 16, the determination unit 17, and the irradiation range controller 18, various processors shown below can be used. The above-described various processors include, for example, a programmable logic device (PLD) which is a processor having a changeable circuit configuration after manufacturing, such as an FPGA, and a dedicated electrical circuit which is a processor having a dedicated circuit configuration designed to execute specific processing, such as an application specific integrated circuit (ASIC), in addition to the GPU and the CPU which is a general-purpose processor that executes software (programs) to function as various processing units, as described above.

One processing unit may be configured with one of the various types of processors, or a combination of two or more processors of the same type or different types (for example, a combination of a plurality of FPGAs, or a combination of a CPU and an FPGA). Further, a plurality of processing units may be configured with one processor.

As an example of configuring a plurality of processing units with one processor, first, there is a form in which, as typified by computers such as a client and a server, one processor is configured by combining one or more CPUs and software, and the processor functions as a plurality of processing units. A second example of the configuration is a form in which a processor that implements the functions of the entire system including a plurality of processing units using one integrated circuit (IC) chip is used. A representative example of this form is a system-on-chip (SoC). As described above, the various types of processing units are configured using one or more of the above-described various types of processors as a hardware structure.

Further, as the hardware structure of these various processors, more specifically, an electric circuit (circuitry), in which circuit elements such as semiconductor elements are combined, can be used.

Moreover, in the above-described embodiments described above, the aspect has been described in which the processing program 110 is stored (installed) in advance in the non-volatile memory 103, but the disclosed technology is not limited to this. The processing program 110 may be provided in a form in which the programs are recorded in a recording medium such as a compact disc read only memory (CD-ROM), a digital versatile disc read-only memory (DVD-ROM), and a universal serial bus (USB) memory. Alternatively, a form may be employed in which the above-described processing program 110 is downloaded from an external device via the network.

Regarding the first to third embodiments, the following appendixes are further disclosed.

### APPENDIX 1

An information processing apparatus comprising at least one processor,
wherein the processor is configured to:
   recognize a specific part of a subject from an optical image obtained by capturing the subject;
   derive a distance to the recognized specific part using a distance image obtained by capturing the subject; and
   derive an irradiation range of radiation at a position corresponding to the derived distance in an irradiation axis direction.

### APPENDIX 2

The information processing apparatus according to appendix 1,
wherein the processor is configured to display information indicating the derived irradiation range on a display screen, by superimposing the information on the optical image.

### APPENDIX 3

The information processing apparatus according to appendix 1 or 2,
wherein the processor is configured to display the optical image and a radiation image side by side.

### APPENDIX 4

The information processing apparatus according to any one of appendixes 1 to 3,
wherein the processor is configured to issue an alert in a case in which the recognized specific part is included in the derived irradiation range.

### APPENDIX 5

The information processing apparatus according to any one of appendixes 1 to 4,
wherein the processor is configured to control the irradiation range of the radiation such that the recognized specific part is not included in the irradiation range of the radiation in accordance with a command, in a case where the recognized specific part is included in the derived irradiation range.

### APPENDIX 6

The information processing apparatus according to appendix 5,
wherein the processor is configured to control the irradiation range of the radiation by controlling an opening amount of an opening portion, through which the radiation passes, of a collimator that forms the opening portion.

### APPENDIX 7

The information processing apparatus according to any one of appendixes 1 to 6,
wherein the specific part is an eyeball of the subject.

### APPENDIX 8

A radiography system comprising:
the information processing apparatus according to any one of appendixes 1 to 7;
an optical camera that generates the optical image;
a distance sensor that generates the distance image; and
a radiation source unit that emits radiation.

### APPENDIX 9

The radiography system according to appendix 8,
wherein the optical camera is disposed outside an irradiation range of the radiation emitted from the radiation source unit.

### APPENDIX 10

The radiography system according to appendix 8 or 9,
wherein the optical camera is disposed at a position closer to an irradiation axis of the radiation emitted from the radiation source unit with respect to the distance sensor.

### APPENDIX 11

The radiography system according to any one of appendixes 8 to 10,
wherein the distance sensor is a ToF camera or a stereo camera.

### APPENDIX 12

An information processing method executed by at least one processor included in an information processing apparatus, the method including a process comprising:
recognizing a specific part of a subject from an optical image obtained by capturing the subject;
deriving a distance to the recognized specific part using a distance image obtained by capturing the subject; and
deriving an irradiation range of radiation at a position corresponding to the derived distance in an irradiation axis direction.

### APPENDIX 13

A program that causes at least one processor included in an information processing apparatus, to execute a process comprising:
recognizing a specific part of a subject from an optical image obtained by capturing the subject;
deriving a distance to the recognized specific part using a distance image obtained by capturing the subject; and
deriving an irradiation range of radiation at a position corresponding to the derived distance in an irradiation axis direction.

What is claimed is:

1. An information processing apparatus comprising at least one processor,
wherein the processor is configured to:
recognize a specific part of a subject from an optical image obtained by capturing the subject;
derive a distance to the recognized specific part using a distance image obtained by capturing the subject; and
derive an irradiation range of radiation at a position corresponding to the derived distance in an irradiation axis direction.

2. The information processing apparatus according to claim 1,
wherein the processor is configured to display information indicating the derived irradiation range on a display screen, by superimposing the information on the optical image.

3. The information processing apparatus according to claim 1,
wherein the processor is configured to display the optical image and a radiation image side by side.

4. The information processing apparatus according to claim 1,
wherein the processor is configured to issue an alert in a case where the recognized specific part is included in the derived irradiation range.

5. The information processing apparatus according to claim 1,
wherein the processor is configured to control the irradiation range of the radiation such that the recognized specific part is not included in the irradiation range of the radiation in accordance with a command, in a case where the recognized specific part is included in the derived irradiation range.

6. The information processing apparatus according to claim 5,
wherein the processor is configured to control the irradiation range of the radiation by controlling an opening amount of an opening portion, through which the radiation passes, of a collimator that forms the opening portion.

7. The information processing apparatus according to claim 1,
wherein the specific part is an eyeball of the subject.

8. A radiography system comprising:
the information processing apparatus according to claim 1;
an optical camera that generates the optical image;
a distance sensor that generates the distance image; and
a radiation source unit that emits radiation.

9. The radiography system according to claim 8,
wherein the optical camera is disposed outside an irradiation range of the radiation emitted from the radiation source unit.

10. The radiography system according to claim 8,
wherein the optical camera is disposed at a position closer to an irradiation axis of the radiation emitted from the radiation source unit with respect to the distance sensor.

11. The radiography system according to claim 8,
wherein the distance sensor is a ToF camera or a stereo camera.

12. An information processing method executed by at least one processor included in an information processing apparatus, the method including a process comprising:
recognizing a specific part of a subject from an optical image obtained by capturing the subject;
deriving a distance to the recognized specific part using a distance image obtained by capturing the subject; and
deriving an irradiation range of radiation at a position corresponding to the derived distance in an irradiation axis direction.

13. A non-transitory computer-readable storage medium storing a program that causes at least one processor included in an information processing apparatus, to execute a process comprising:
recognizing a specific part of a subject from an optical image obtained by capturing the subject;
deriving a distance to the recognized specific part using a distance image obtained by capturing the subject; and
deriving an irradiation range of radiation at a position corresponding to the derived distance in an irradiation axis direction.
